(19) United States
(12) Patent Application Publication
(10) Pub. No.: US 2018/0295971 A1
(43) Pub. Date: Oct. 18, 2018

(54) COSMETIC SHEET

(71) Applicant: AMOLIFESCIENCE CO., LTD., Seoul (KR)

(72) Inventors: In Yong SEO, Seoul (KR); Ji Hyun LEE, Incheon (KR)

(21) Appl. No.: 15/768,260

(22) PCT Filed: Oct. 17, 2016
(86) PCT No.: PCT/KR2016/011613

§ 371 Date: Apr. 13, 2018

(30) Foreign Application Priority Data
Oct. 16, 2015 (KR) 10-2015-0144639

(51) Int. Cl. A45D 44/22; A61K 8/02; A61K 8/66

(52) U.S. Cl. CPC A45D 44/22; A61K 8/66; A61K 8/0212

(57) ABSTRACT

Provided is a cosmetic sheet, including a support and a membrane, which is provided on the support, is attached to the skin, and is formed by arranging a fiber containing a water-soluble polymer, a water-insoluble polymer, and a functional material for skin care that is released by the dissolution of the water-soluble polymer.

### TECHNICAL FIELD

The present invention relates to a cosmetic sheet, and more particularly to a dry cosmetic sheet.

### BACKGROUND ART

Recently, interest in skin care is continuously increasing.

Skin care has been conventionally carried out mainly for women who are active outdoors, but is nowadays performed not only by general housewives but also by adult men.

With increasing age, wrinkles appear on the face and the elasticity of the skin decreases. In particular, women are making great efforts to prevent skin aging as a natural way of pursuing beauty, and even adult men tend to be concerned with skin care in order to improve their interpersonal relations and social life.

Cosmetic packs that are easily used for such skin care have been developed and are currently available. However, conventional cosmetic packs, which are manufactured from a woven or nonwoven fabric, are problematic in that a liquid essence that is added may flow down due to its intrinsic weight and the use thereof is inconvenient due to the flowing down of excess essence.

In this regard, Korean Patent Application Publication No. 10-2010-0104790 (Patent Document 1) discloses a hand/foot care pack, comprising a sterile absorbent mitten in which fiber sheet layers are superimposed on each other and adhered along the outer circumference of the mitten so that the fiber layer possesses a cosmetic material and is sterilized, a cosmetic material present in the state in which the sterile absorbent mitten is wet, and a hermetic packing bag in which the mitten wet with the cosmetic material is packed hermetically for transfer to a consumer.

Such a hand/foot care pack is problematic because the liquid cosmetic material wets the fiber sheet layers and may thus flow down.

Furthermore, in the case of a liquid facial care pack, including the above liquid hand/foot care pack, a typical functional material that is not degenerated but may be stored even in a liquid phase for a long period of time may be loaded, but a functional material that is difficult to store in a liquid phase for a long period of time, for example, a vitamin, an enzyme, a protein, or a peptide-vitamin C derivative, cannot be used because it is degenerated when stored in a liquid phase for a long period of time, making it impossible to provide a highly functional product desired by consumers.

A conventional cosmetic pack is mostly configured such that a substrate such as a woven or nonwoven fabric is wetted with a liquid material containing a functional material and then packaged. Here, the functional material is used in a large amount, and a highly functional material such as a vitamin, an enzyme, a protein, or a peptide-vitamin C derivative is expensive, and the use thereof is limited. Cosmetic packs using such a highly functional material have not been provided in the form of a product that has been adopted in the real world.

Furthermore, the highly functional material, which is expensive, is added in a very small amount to a liquid material (e.g. a liquid cosmetic) including a typical functional material, and is not uniformly loaded when a substrate such as a nonwoven fabric is impregnated therewith. A surplus liquid cosmetic that is packaged together with the nonwoven fabric substrate in a pouch is not used but is discarded in most cases, and thus the highly functional material, which is expensive, contained therein, is not effectively used.

Accordingly, there is a need to develop a novel type of cosmetic pack or sheet, which may solve the problems with conventional liquid cosmetic packs.

### DISCLOSURE

### Technical Problem

The present invention has been made keeping in mind the problems encountered in the related art, and is intended to provide a cosmetic sheet, in which a functional material that is difficult to store in a liquid phase for a long period of time may be incorporated in a dry state into a fiber that constitutes a dry sheet, thereby enabling long-term storage thereof and increasing a skin-care effect.

In addition, the present invention is intended to provide a cosmetic sheet, in which a highly functional material is uniformly loaded by forming a membrane through an electrospinning process using a polymer spinning solution including a small amount of the highly functional material, which is expensive and difficult to store in a liquid phase for a long period of time.

In addition, the present invention is intended to provide a cosmetic sheet, in which the cosmetic sheet containing a highly functional material is packed and stored in a dry state and is then used by being wet using a wetting agent or water, whereby the highly functional material is not degenerated even upon long-term storage.

In addition, the present invention is intended to provide a cosmetic sheet, in which a functional material is loaded in the cosmetic sheet using an electrospinning process, whereby the functional material, which is expensive, may be uniformly distributed throughout the cosmetic sheet even when used in a very small amount, thus realizing low-cost production.

In addition, the present invention is intended to provide a cosmetic sheet, in which a nanofiber having a core-shell structure is formed through electrospinning of a water-soluble polymer and a water-insoluble polymer, and thus, when the cosmetic sheet is used, the water-soluble polymer, corresponding to the shell, is dissolved by a wetting agent and thus the functional material is released, and the nanofiber composed of the water-insoluble polymer corresponding to the core may maintain its three-dimensional network, thus preventing the flowing down of the functional material.

In addition, the present invention is intended to provide a cosmetic sheet, in which only a dry cosmetic sheet including a nonwoven fabric substrate is packaged in a pouch, and a surplus liquid cosmetic is not placed in the pouch, thus reducing the waste of the liquid cosmetic that is not used but is discarded and lowering manufacturing costs.

In addition, the present invention is intended to provide a cosmetic sheet, which is in an ultrathin film form and appears transparent when attached to the face using a wetting agent and the attachment of which may be maintained, whereby it may be easily worn even upon outdoor activities such as climbing or exercises.

In addition, the present invention is intended to provide a cosmetic sheet, in which a shape-retaining layer is transparent and changes to a white color when dried, thus easily checking the time point at which the cosmetic sheet is removed to thereby realize convenient use thereof.

### Technical Solution

Therefore, an embodiment of the present invention provides a cosmetic sheet, comprising: a support; and a membrane, which is provided on the support, is attached to the skin, and is formed by arranging a fiber containing a water-soluble polymer, a water-insoluble polymer, and a functional material for skin care, which is released by the dissolution of the water-soluble polymer.

Here, the cosmetic sheet of the present invention may be provided at a predetermined size in the form of being attached to the entire face, hands, feet, portions of the face under the eyes, nose, neck or arms, and may then be dry-packed in a pouch. It may be used by spraying a wetting agent such as an essence or water onto the membrane or by spraying a wetting agent onto the skin and then attaching the cosmetic sheet.

According to the present invention, the cosmetic sheet including the membrane is very thin and lightweight, and may thus be effectively attached to the skin at a desired region using a small amount of wetting agent and resists detachment.

The membrane of the present invention is transparent, and may solve the problem in that a conventional opaque cosmetic sheet is not visually attractive. By virtue of the high adhesion and transparency, the present invention may provide products with high customer satisfaction because the cosmetic sheet may be easily worn even upon outdoor activities, such as climbing or walking.

The functional material may include a dry storage material that is difficult to store in a liquid phase for a long period of time, and the dry storage material may be any one selected from among a vitamin, an enzyme, a protein, and a peptide-vitamin C derivative.

The fiber may further include a fiber containing the water-insoluble polymer and the functional material, and/or a fiber containing the water-soluble polymer and the functional material.

The fiber may have a core-shell structure configured such that the water-insoluble polymer is disposed at a core and such that the water-soluble polymer is disposed around the water-insoluble polymer.

In the fiber having the core-shell structure, the water-soluble polymer disposed outside is dissolved by the wetting agent, and the core comprising the water-insoluble polymer disposed inside may maintain its shape, whereby the shape of the membrane may be maintained.

The support may be formed of any one selected from among a nonwoven fabric, a mesh, silicon, a PET film, a PE film, a PP film, and a PU film.

Also, a release film for protecting the membrane may be attached to the membrane.

Also, the membrane may include a shape-retaining layer, provided on the support and formed by arranging a first fiber containing the water-insoluble polymer, and a skin contact layer, formed by arranging, on the shape-retaining layer, a second fiber containing the water-soluble polymer and the functional material.

Here, the shape-retaining layer may comprise a nanofiber web having a three-dimensional network structure having micropores formed by arranging, on the support, the first fiber obtained through electrospinning of a spinning solution comprising the water-insoluble polymer and a solvent, which are mixed together.

The skin contact layer may comprise a nanofiber web having a three-dimensional network structure having micropores formed by arranging, on the shape-retaining layer, the second fiber obtained through electrospinning of a spinning solution comprising the water-soluble polymer, the functional material for skin care including the dry storage material, which is difficult to store in a liquid phase, and a solvent, which are mixed together.

Each of the first fiber and the second fiber may have a diameter of less than 3 μm.

The shape-retaining layer is transparent when a wetting agent is contained therein, and may change to a color that is visible when the wetting agent is dried.

When the fiber contains the water-insoluble polymer, the functional material and the water-soluble polymer, the water-soluble polymer and the water-insoluble polymer may be mixed at a weight ratio ranging from 3:7 to 9:1. In this case, the water-soluble polymer and the water-insoluble polymer are preferably mixed at a weight ratio of 7:3.

Also, the functional material may be used in an amount of 0.1 to 15 wt % based on the total weight of the polymer. The functional material includes a vitamin, and the vitamin may be used in an amount of 5 to 10 wt % based on the total weight of the polymer.

The membrane may have a thickness ranging from 5 to 10 μm and a basis weight ranging from 3 to 30 gsm.

When the membrane includes the shape-retaining layer and the skin contact layer, the shape-retaining layer may have a thickness ranging from 5 to 10 μm and a basis weight ranging from 3 to 20 gsm, and the skin contact layer may have a thickness ranging from 2 to 5 μm and a basis weight ranging from 1 to 10 gsm.

In the cosmetic sheet of the present invention, the exposed surface of the shape-retaining layer may be provided with a printed layer for beautifying a design thereof.

The fiber of the cosmetic sheet has a core-shell structure, and is transparent when attached to the skin using the wetting agent, and the water-soluble polymer disposed outside the fiber is dissolved by the wetting agent, and the core comprising the water-insoluble polymer disposed inside maintains its shape, whereby the shape of the membrane may be maintained.

### Advantageous Effects

According to the present invention, a dry cosmetic sheet composed of a fiber in which a dry storage material that is difficult to store in a liquid phase for a long period of time is incorporated can be provided, whereby the dry storage material that decomposes in a liquid phase, such as a vitamin, an enzyme, a protein, or a peptide-vitamin C derivative, can be stored for a long period of time, and the skin-care effect thereof can be maximized.

Also, according to the present invention, a highly functional material, which is expensive and difficult to store in a liquid phase for a long period of time, is mixed in a very small amount with a polymer spinning solution and then electrospun, thus forming a membrane, whereby a cosmetic sheet in which the highly functional material is uniformly loaded can be provided.

Furthermore, when the functional material is loaded in the cosmetic sheet through an electrospinning process, a very small amount of a functional material, which is expensive, can be uniformly distributed throughout the cosmetic sheet, thus enabling low-cost production.

In the present invention, only the dry cosmetic sheet including a nonwoven fabric substrate is packaged in a pouch, and a surplus liquid cosmetic is not placed in the pouch, thus reducing the waste of the liquid cosmetic that is not used but is discarded and lowering manufacturing costs.

In the present invention, a nanofiber having a core-shell structure is obtained through electrospinning of a water-soluble polymer and a water-insoluble polymer, and thus, upon using the cosmetic sheet, the water-soluble polymer corresponding to the shell is dissolved by the wetting agent and the functional material is released, and the nanofiber composed of the water-insoluble polymer corresponding to the core maintains its three-dimensional network, thereby preventing the flowing down of the functional material.

According to the present invention, when the water-soluble polymer is dissolved by the wetting agent, the peptide-vitamin C derivative having high skin penetration capability is released, thereby promoting the penetration of the functional material into the skin.

According to the present invention, the shape-retaining layer, which is transparent, turns to a white color when dried, whereby the time point for removal of the cosmetic sheet can be determined, thus being convenient to the user.

The cosmetic sheet of the present invention has an ultrathin-film sheet structure. When it is attached to the skin, such as the face or the like, using the wetting agent, it appears transparent and the attachment thereof can be maintained, whereby it can be worn easily even upon outdoor activities such as climbing or exercises.

### BRIEF DESCRIPTION OF DRAWINGS

FIG. 1 is a cross-sectional view showing a cosmetic sheet according to a first embodiment of the present invention;

FIGS. 2a to 2c show various shapes of the cosmetic sheet according to the present invention;

FIG. 3 is a cross-sectional view showing the fiber of the membrane of the cosmetic sheet according to the first embodiment of the present invention;

FIG. 4 is a cross-sectional view showing a cosmetic sheet according to a second embodiment of the present invention; and

FIG. 5 is a cross-sectional view showing a release film formed on the cosmetic sheet according to the first and second embodiments of the present invention.

### MODE FOR INVENTION

Hereinafter, a detailed description will be given of embodiments of the present invention with reference to the accompanying drawings.

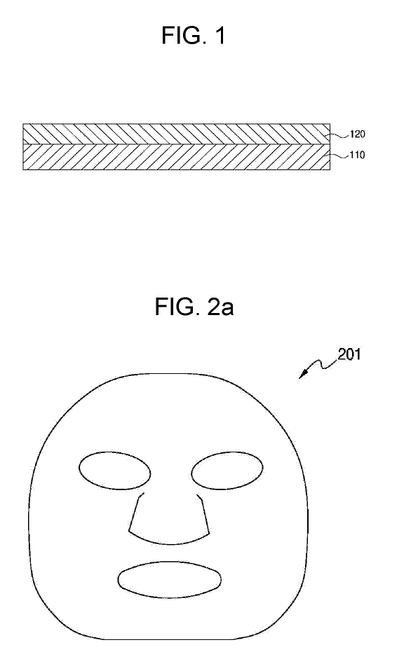

As illustrated in FIG. 1, a cosmetic sheet 100 according to a first embodiment of the present invention comprises a support 110; and a membrane 120, which is provided on the support 110, is attached to the skin, and is formed by arranging a fiber containing a water-soluble polymer, a water-insoluble polymer, and a functional material for skin care including a dry storage material that is released by the dissolution of the water-soluble polymer and is difficult to store in a liquid phase for a long period of time. Here, the dry storage material is a functional material for which dry storage is suitable because it is difficult to store in a liquid phase.

The cosmetic sheet 100 of the present invention is attached to the skin of the body to thus enhance the physiological function of the skin, stimulate the metabolism, and supply nutrition, and may thereby be utilized as a sheet that can keep the skin beautiful, healthy and elastic.

The cosmetic sheet 100 may be made into a shape suitable for the body structure through processing such as cutting, incision, punching, or folding. The cosmetic sheet 100 may be manufactured to have a size and shape suitable for attachment to all parts of the body such as the face, neck, hands, ears, legs, abdomen, arms, and shoulders.

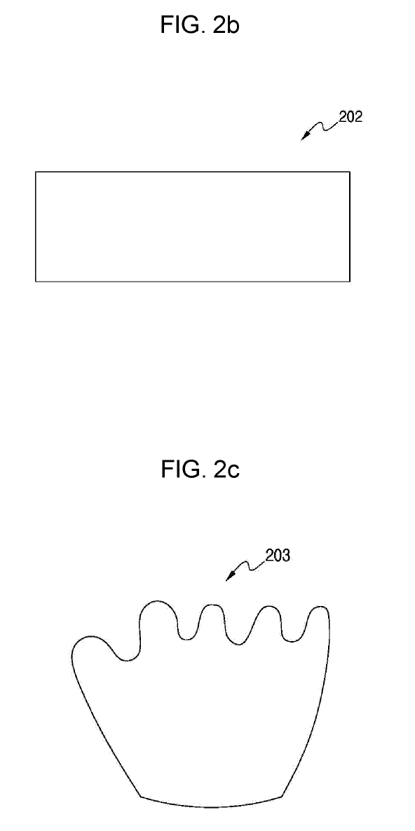

For example, as shown in FIGS. 2a to 2c, the cosmetic sheet 100 may be provided in the form of a facial cosmetic sheet 201 suitable for the face, a cosmetic sheet 202 having a size that may be attached to any region of the body, or a cosmetic sheet 203 suitable for the back of the hand.

The dry storage material may be a vitamin, an enzyme, a protein, or a peptide-vitamin C derivative, which decomposes in a liquid phase and is thus difficult to store in a liquid phase.

The dry storage material also includes a typical functional material, in addition to the material that is difficult to store in a liquid phase for a long period of time.

Therefore, in the present invention, the dry storage material that is difficult to store in a liquid phase for a long period of time is contained in the fiber of the membrane 120 of the cosmetic sheet 100 and thus enables long-term storage in a dry state incorporated into the fiber. Also, in the present invention, when the water-soluble polymer is dissolved by the wetting agent, the functional material is released and transferred to the skin, thereby maximizing the skin-care effect.

Specifically, when the cosmetic sheet 100 of the present invention is attached to the skin, the water-soluble polymer of the membrane 120 is dissolved by the wetting agent so that the functional material and the water-soluble polymer are released from the fiber. The released functional material is absorbed into the skin, and the water-soluble polymer dissolved in the wetting agent is absorbed into the support 110. The water-insoluble polymer is not dissolved in the wetting agent but is able to maintain the shape of the membrane. Here, the shape of the membrane is maintained but may change before and after the release of the functional material due to the dissolution of the water-soluble polymer.

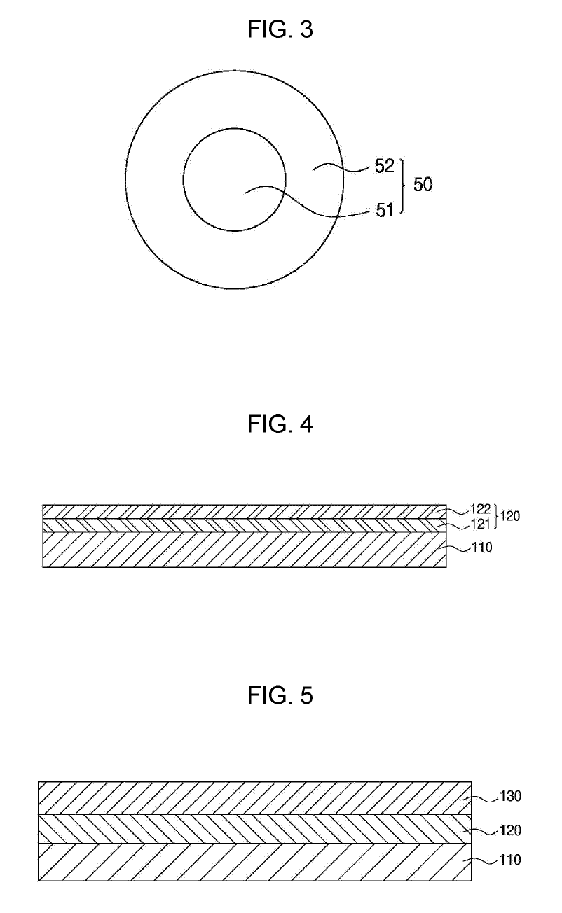

As will be described later, the fiber 50 of the cosmetic sheet may have a core-shell structure configured such that the water-insoluble polymer 51 is disposed at the core and the water-soluble polymer 52 is disposed around the water-insoluble polymer 51 (FIG. 3). Thus, the water-soluble polymer 52 positioned outside the fiber is dissolved by the wetting agent and the core comprising the water-insoluble polymer 51 positioned inside maintains its shape, whereby the shape of the membrane is maintained.

Meanwhile, the peptide-vitamin C derivative is a peptide to which vitamin C is linked, and is specifically configured such that vitamin C is linked to a peptide, which is a polymer composed of 10 or less amino acids connected through peptide bonding.

The peptide-vitamin C derivative has a structure comprising a small number of bonded amino acids and thus facilitates penetration into the skin.

In the cosmetic sheet 100 of the present invention, when the water-soluble polymer is dissolved, the peptide-vitamin C derivative and the functional material may be released together, whereby the functional material may rapidly penetrate into the skin, thus obtaining a superior skin-care effect.

The membrane 120 is formed in a manner in which a water-soluble polymer, a water-insoluble polymer, a functional material including a dry storage material that is difficult to store in a liquid phase, and a solvent are mixed at an appropriate ratio to give a spinning solution, which is then electrospun to form a fiber having a diameter of less than 3 μm, which is then arranged into a fiber web.

The water-soluble polymer is a fiber-forming polymer that may be subjected to electrospinning, and is dissolved in a wetting agent, and may include any one or a mixture of two or more selected from among PVA (polyvinyl alcohol), PVP (polyvinyl pyrrolidone), PEO (polyethylene oxide), CMC (carboxyl methyl cellulose), starch, PAA (polyacrylic acid), and hyaluronic acid.

The functional material is used for skin care, and may include any one or a mixture of two or more selected from among ingredients for skin whitening (arbutin, niacinamide, ascorbyl glucoside), ingredients for skin anti-wrinkling (retinol, adenosine), ingredients for UV protection (titanium dioxide), and ingredients for moisturization and skin elasticity (snail mucilage filtrate, acetyl hexapeptide, red ginseng collagen, aqua ceramide, regenerating peptide, and Galactomyces fermentation broth).

The functional material may include any one or a mixture of two or more selected from among water-soluble collagen, vegetable platina, tocopherol, xylitol, and plant extracts.

The water-insoluble polymer is a fiber-forming polymer that may be subjected to electrospinning in order to form the structure of the membrane, and functions to maintain the structure of the membrane 120, regardless of whether the functional material is released by the dissolution of the water-soluble polymer in the wetting agent. As the water-insoluble polymer, any resin may be used without particular limitation so long as it may be dissolved in a solvent so as to perform electrospinning and may be formed into a nanofiber through electrospinning. Examples thereof may include polyvinylidene fluoride (PVdF), poly(vinylidene fluoride-co-hexafluoropropylene), perfluoropolymer, polyvinyl chloride, polyvinylidene chloride or copolymers thereof, polyethylene glycol derivatives including polyethylene glycol dialkyl ether and polyethylene glycol dialkyl ester, polyoxides including poly(oxymethylene-oligo-oxyethylene), polyethylene oxide and polypropylene oxide, polyvinyl acetate, poly(vinyl pyrrolidone-vinyl acetate), polystyrene and polystyrene acrylonitrile copolymers, polyacrylonitrile copolymers including polyacrylonitrile (PAN) and polyacrylonitrile methyl methacrylate copolymers, polymethyl methacrylate, polymethylmethacrylate copolymers, or mixtures thereof.

The solvent may include water or an alcohol, and an organic solvent may also be used in addition to the water or alcohol.

The support 110 functions to support the membrane 120, and may be a nonwoven fabric. The support 110 may include any type of material able to support the cosmetic pack, such as silicon, PE, PP, PU film, etc., in addition to the nonwoven fabric.

The support 110 may be a mesh that is able to support the entire cosmetic sheet while having a beautiful design. The mesh is formed by weaving a silver yarn, a fiber yarn, an aluminum wire, or a resin yarn.

When the cosmetic sheet 100 according to the first embodiment of the present invention has a two-layer structure comprising the support 110 and the membrane 120, the membrane 120 may have a thickness ranging from 5 to 10 μm, with a maximum of 20 μm, and a basis weight of 3 to 30 gsm. The support 110 may have a thickness ranging from 100 to 400 μm, and a basis weight of 30 to 300 gsm.

If the thickness of the membrane 120 is less than 5 μm, handling becomes difficult due to low strength. On the other hand, if the thickness thereof exceeds 10 μm, transparency may be lost, and the increase in efficacy is not proportional to the increase in the product cost. Also, if the basis weight of the membrane 120 is less than 3 gsm, handling becomes difficult due to low strength. On the other hand, if the basis weight thereof exceeds 30 gsm, transparency may be lost, and the manufacturing costs may increase.

In the method of manufacturing the cosmetic sheet 100 according to the first embodiment of the present invention, the spinning solution comprising the water-insoluble polymer, the water-soluble polymer and the functional material is electrospun to give a fiber, after which the fiber thus obtained is arranged, resulting in a membrane 120 having a fiber web structure.

Here, a fiber containing the water-insoluble polymer and the functional material, a fiber containing the water-soluble polymer and the functional material, and a fiber containing the water-insoluble polymer, the functional material and the water-soluble polymer may be obtained through electrospinning.

The membrane 120 may be formed of only the first fiber comprising the water-insoluble polymer, the water-soluble polymer and the functional material, and the membrane 120 may have a stacked structure comprising at least two selected from among a second fiber containing a water-insoluble polymer and a functional material, a third fiber containing a water-soluble polymer and a functional material, a first fiber containing a water-insoluble polymer, a water-soluble polymer and a functional material.

When the fiber contains all of the water-insoluble polymer, the functional material and the water-soluble polymer, the water-soluble polymer and the water-insoluble polymer are mixed at a weight ratio of 3:7 to 9:1, and the water-soluble polymer and the water-insoluble polymer are preferably mixed at a weight ratio of 7:3.

The functional material may be used in an amount of 0.1 to 15 wt % based on the total weight of the polymer. In particular, a vitamin may be used in an amount of 5 to 10 wt % based on the total weight of the polymer.

During the mixing of the water-soluble polymer and the water-insoluble polymer, if the amount of the water-soluble polymer is less than 30 wt %, wetting with the wetting agent is impossible. On the other hand, if the amount thereof exceeds 90 wt %, the web is not formed upon electrospinning the spinning solution in order to obtain the membrane 120 having the fiber web structure.

If the amount of the functional material is less than 0.1 wt % based on the total weight of the polymer, it is difficult to exhibit desired performance due to the small amount thereof. On the other hand, if the amount thereof exceeds 15 wt %, the spinning solution cannot be electrospun and costs may increase.

With reference to FIG. 3, the fiber 50 of the cosmetic sheet 100 according to the first embodiment of the present invention is configured such that the water-insoluble polymer 51 is disposed at the core and the water-soluble polymer 52 is disposed around the water-insoluble polymer 51. Since the water-insoluble polymer 51 and the water-soluble polymer 52 are different from each other, when the electrospinning is performed, a core-shell structure in which the water-insoluble polymer 51 is disposed at the core and the water-soluble polymer 52 is disposed around the water-insoluble polymer 51 may result.

Here, the core-shell structure may be formed because the water-insoluble polymer 51 and the water-soluble polymer 52 are different from each other in terms of molecular weight or melting point.

With reference to FIG. 4, the membrane 120 of a cosmetic sheet according to a second embodiment of the present invention is formed on the support 110, and may include a shape-retaining layer 121 formed by arranging a first fiber containing a water-insoluble polymer and a skin contact layer 122 formed by arranging, on the shape-retaining layer 121, a second fiber containing a water-soluble polymer and a functional material including a dry storage material that is difficult to store in a liquid phase.

When the membrane 120 of the cosmetic sheet according to the second embodiment of the present invention has a two-layer structure comprising the shape-retaining layer 121 and the skin contact layer 122, the shape-retaining layer 121 may have a thickness of 5 to 10 μm and a basis weight of 3 to 20 gsm. Also, the skin contact layer 122 may have a thickness of 2 to 5 μm and a basis weight of 1 to 10 gsm.

If the thickness of the shape-retaining layer 121 is less than 5 μm and the basis weight thereof is less than 3 gsm, handling is difficult due to the low strength thereof. On the other hand, if the thickness of the shape-retaining layer 121 exceeds 10 μm and the basis weight thereof exceeds 30 gsm, transparency may be lost.

Also, if the thickness of the skin contact layer 122 is less than 2 μm and the basis weight thereof is less than 1 gsm, it is difficult to exhibit desired performance due to the small amount of functional material. On the other hand, if the thickness of the skin contact layer 122 exceeds 5 μm and the basis weight thereof exceeds 10 gsm, the amount of the water-soluble polymer may increase and thus a phenomenon of excessive stickiness may occur.

The shape-retaining layer 121 is composed of a nanofiber web having a three-dimensional network structure having micropores obtained by mixing a water-insoluble polymer that enables electrospinning and a solvent to give a spinning solution, which is then electrospun to form a first fiber, and then arranging the first fiber on the support 110.

The skin contact layer 122 is composed of a nanofiber web having a three-dimensional network structure having micropores obtained by mixing a water-soluble polymer that enables electrospinning, a functional material including a dry storage material that is difficult to store in a liquid phase, and a solvent to give a spinning solution, which is then electrospun to form a second fiber, and then arranging the second fiber on the shape-retaining layer 121.

In the second fiber of the skin contact layer 122, the functional material may be contained in an amount of 0.1 to 15 wt % based on the amount of the water-soluble polymer. Furthermore, the vitamin is preferably contained in an amount of 5 to 10 wt % based on the amount of the water-soluble polymer.

As such, if the amount of the functional material mixed with the water-soluble polymer is less than 0.1 wt %, it is difficult to exhibit desired performance due to the small amount thereof. On the other hand, if the amount thereof exceeds 15 wt %, the spinning solution cannot be electrospun and costs may increase.

The first fiber of the shape-retaining layer 121 and the second fiber of the skin contact layer 122 may be designed to have the same diameter or different diameters. As such, the diameter of each of the first fiber and the second fiber may be less than 3 μm.

The shape-retaining layer 121 functions to maintain the shape of the membrane 120 when the membrane 120 is attached to the skin and to maintain the moisturizing effect by absorbing an essence that is sprayed on the skin into the micropores defined by the first fiber.

The shape-retaining layer 121 is provided in the form of a fiber web by arranging the fiber obtained through electrospinning of the spinning solution including the water-insoluble polymer. In the state in which the essence is absorbed, that is, in which the wetting agent is contained, diffuse reflection does not occur and thus it consequently appears transparent. On the other hand, in an essence-free state, namely in the state in which the wetting agent is dried, a white color or a color other than the white color appears, and thus the thorough absorption of the skin contact layer 122 into the skin may be visually confirmed.

When the functional material of the skin contact layer 122 is thoroughly absorbed into the skin and thus the wetting agent is dried, the shape-retaining layer 121 turns white and the time point at which the cosmetic sheet is removed may be checked visually by the user.

Meanwhile, a predetermined period of time after the attachment of a conventional cosmetic sheet to the skin, the cosmetic sheet is stripped from the skin. Here, persons have different time periods for which the material of the cosmetic sheet is absorbed into the skin. Hence, when the cosmetic sheet is removed after the designated time period, there may occurs cases where the sheet is removed despite the functional material contained in the cosmetic sheet not being thoroughly absorbed into the skin, or where the sheet is still attached to the skin despite the functional material being thoroughly absorbed into the skin.

The shape-retaining layer 121 of the present invention, which is transparent, changes to a white color when the wetting agent is dried, and thus the time point at which the cosmetic sheet is removed from the skin may be checked visually, thus realizing convenient use.

As the wetting agent, any material may be used without limitation so long as it is safe enough not to cause skin problems while the water-soluble polymer of the skin contact layer 122 is dissolving. Preferably useful as the wetting agent is water, an essence, a mist, or the like.

Provided on one surface of the shape-retaining layer 121 may be a printed layer (not shown) for realizing an advertisement pattern or beautifying the design.

When the skin contact layer 122 including the mixture of the water-soluble polymer and the functional material is attached to the skin, the functional material is absorbed into the skin and the water-soluble polymer is absorbed into the support 110, thus achieving a moisturizing effect.

The solvent contained in the spinning solution for forming the skin contact layer 122 preferably includes water or an alcohol, and may prevent skin problems from occurring due to the use of an organic solvent.

Also, a printed layer for beautifying a design may be formed on one surface of the shape-retaining layer 121.

With reference to FIG. 5, the cosmetic sheet according to the first and second embodiments of the present invention may include a release film 130 that is formed on the membrane 120 and separated upon use of the cosmetic sheet.

The release film 130 functions to protect the membrane and is formed of PET, PP, PE or release paper and has strength suitable for sufficiently supporting the membrane 120.

As described above, in the cosmetic sheet of the present invention, the highly functional material that is expensive and difficult to store in a liquid phase for a long period of time, such as a vitamin, an enzyme, a protein, a peptide-vitamin C derivative, etc., is mixed in a small amount with the polymer spinning solution, followed by electrospinning, thus obtaining a cosmetic sheet in which the highly functional material is uniformly loaded, which is then packed and stored in a dry state, and is applied by being wet using a wetting agent or water, whereby the functional material is not degenerated even upon long-term storage, thanks to the dry storage of the highly functional material. The cosmetic sheet may also contain a typical functional material.

The cosmetic sheet of the present invention including the membrane is very thin and lightweight and may thus be strongly attached to the skin at a desired region using a small amount of wetting agent and resists detachment. Furthermore, the membrane of the present invention appears transparent, and may solve the problems with a conventional opaque cosmetic sheet which is not visually attractive. By virtue of the high adhesion and transparency, the present invention may provide products with high customer satisfaction because the cosmetic sheet may be easily worn even upon outdoor activities such as climbing or walking.

Moreover, in the present invention, the functional material is loaded in the cosmetic sheet using an electrospinning process, and thereby, even when the expensive functional material is used in a very small amount, the functional material is uniformly distributed throughout the cosmetic sheet, thus enabling low-cost production.

Although the preferred embodiments of the present invention have been disclosed for illustrative purposes, those skilled in the art will appreciate that various modifications and substitutions are possible, without departing from the scope and spirit of the invention as disclosed in the accompanying claims.

### INDUSTRIAL APPLICABILITY

According to the present invention, a dry cosmetic sheet is configured such that a functional material for dry storage, which is difficult to store in a liquid phase, is contained in a fiber, thus enabling the long-term storage thereof and maximizing a skin-care effect.

1. A cosmetic sheet, comprising:
a support; and
a membrane, which is provided on the support, is attached to a skin, and is formed by arranging a fiber containing a water-soluble polymer, a water-insoluble polymer, and a functional material for skin care that is released by dissolution of the water-soluble polymer.

2. The cosmetic sheet of claim 1, wherein the functional material includes a dry storage material that is difficult to store in a liquid phase for a long period of time, and the dry storage material is any one selected from among a vitamin, an enzyme, a protein, and a peptide-vitamin C derivative.

3. The cosmetic sheet of claim 1, wherein the fiber further includes a fiber containing the water-insoluble polymer and the functional material and/or a fiber containing the water-soluble polymer and the functional material.

4. The cosmetic sheet of claim 1, wherein the fiber has a core-shell structure configured such that the water-insoluble polymer is disposed at a core and the water-soluble polymer is disposed around the core comprising the water-insoluble polymer.

5. The cosmetic sheet of claim 1, wherein the support comprises any one selected from among a nonwoven fabric, a mesh, silicon, a PET film, a PE film, a PP film, and a PU film.

6. The cosmetic sheet of claim 1, further comprising a release film attached to an exposed surface of the membrane to protect the membrane.

7. The cosmetic sheet of claim 1, wherein the membrane comprises:
a shape-retaining layer provided on the support and formed by arranging a first fiber containing the water-insoluble polymer; and
a skin contact layer formed by arranging, on the shape-retaining layer, a second fiber containing the water-soluble polymer and the functional material.

8. The cosmetic sheet of claim 7, wherein the shape-retaining layer comprises a nanofiber web having a three-dimensional network structure having micropores formed by arranging, on the support, the first fiber obtained through electrospinning of a spinning solution comprising the water-insoluble polymer and a solvent, which are mixed together, and thus exhibits a moisturizing function.

9. The cosmetic sheet of claim 7, wherein the skin contact layer comprises a nanofiber web having a three-dimensional network structure having micropores formed by arranging, on the shape-retaining layer, the second fiber obtained through electrospinning of a spinning solution comprising the water-soluble polymer, the functional material for skin care including the dry storage material that is difficult to store in the liquid phase, and a solvent, which are mixed together.

10. The cosmetic sheet of claim 7, wherein each of the first fiber and the second fiber has a diameter of less than 3 μm.

11. The cosmetic sheet of claim 7, wherein the shape-retaining layer is transparent when a wetting agent is contained, and changes to a color that is visible when the wetting agent is dried.

12. The cosmetic sheet of claim 1, wherein when the fiber contains the water-insoluble polymer, the functional material and the water-soluble polymer, the water-soluble polymer and the water-insoluble polymer are mixed at a weight ratio ranging from 3:7 to 9:1.

13. The cosmetic sheet of claim 12, wherein the water-soluble polymer and the water-insoluble polymer are mixed at a weight ratio of 7:3.

14. The cosmetic sheet of claim 12, wherein the functional material is used in an amount of 0.1 to 15 wt % based on a total weight of the polymer.

15. The cosmetic sheet of claim 14, wherein the functional material includes a vitamin, and the vitamin is used in an amount of 5 to 10 wt % based on the total weight of the polymer.

16. The cosmetic sheet of claim 4, wherein in the fiber having the core-shell structure, the water-soluble polymer disposed outside is dissolved by a wetting agent and the core comprising the water-insoluble polymer disposed inside maintains a shape thereof so that a shape of the membrane is maintained.

17. The cosmetic sheet of claim 1, wherein the membrane has a thickness ranging from 5 to 10 μm and a basis weight ranging from 3 to 30 gsm.

18. The cosmetic sheet of claim 7, wherein the shape-retaining layer has a thickness ranging from 5 to 10 μm and a basis weight ranging from 3 to 20 gsm, and
the skin contact layer has a thickness ranging from 2 to 5 μm and a basis weight ranging from 1 to 10 gsm.

19. The cosmetic sheet of claim 7, wherein the exposed surface of the shape-retaining layer is provided with a printed layer for beautifying a design thereof.

20. The cosmetic sheet of claim 1, wherein the fiber of the cosmetic sheet has a core-shell structure, and is transparent when attached to the skin using a wetting agent, and
the water-soluble polymer disposed outside the fiber is dissolved by the wetting agent, and the core comprising the water-insoluble polymer disposed inside maintains a shape thereof so that a shape of the membrane is maintained.
